(19) United States
(12) Patent Application Publication
(10) Pub. No.: US 2013/0023656 A1
(43) Pub. Date: Jan. 24, 2013

(54) METHOD FOR SELECTIVE ISOLATION AND PURIFICATION OF NUCLEIC ACIDS

(71) Applicants: Jörg Hucklenbroich, Hilden (DE); Mario Scherer, Hilden (DE)

(72) Inventors: Jörg Hucklenbroich, Hilden (DE); Mario Scherer, Hilden (DE)

(73) Assignee: Qiagen GmbH, Hilden (DE)

(21) Appl. No.: 13/639,806

(22) PCT Filed: Apr. 8, 2011
(86) PCT No.: PCT/EP2011/055549

§ 371 Date: Oct. 5, 2012

(30) Foreign Application Priority Data
Apr. 8, 2010 (EP) 10003767.0

(51) Int. Cl. C07H 1/08

(52) U.S. Cl. 536/25.41

(57) ABSTRACT

The present invention relates to a gentle method for isolating and purifying nucleic acids from a biological cell comprising sample comprising at least DNA, RNA, and proteins, comprising at least the steps of 1. mixing the sample with a lysis buffer, 2. incubating the sample at a temperature within a range between 45° C. and 59° C. to obtain DNA as well as RNA or within a range between 60° C. and 70° C., to obtain DNA essentially free of RNA and 3. separating the nucleic acids from any contaminants.

The isolation of high-quality nucleic acids is a prerequisite for many different techniques in modern molecular biology, such as PCR amplification, blotting analysis and genomic-library construction, used for example in the field of molecular diagnostics. Especially if the nucleic acids are obtained from biological samples containing cellular material, it is necessary to separate them from contaminants like proteins, lipids and other cellular constituents that otherwise may interfere with restriction enzymes, ligases, and/or thermostable DNA polymerases used in these downstream applications. Furthermore, RNA nucleases (RNases) and DNA nucleases (DNases) present in biological samples have to be removed to prevent degradation of the nucleic acids.

A variety of different methods have been developed for the isolation of nucleic acids from biological samples containing cellular components. All of these methods involve a step of disrupting and lysing the starting material by breaking the cellular membrane and releasing its contents into solution. The mixture obtained is called lysate. In the following steps proteins, in particular nucleases, and the contaminants of the mixture and/or the used solutions are removed from the lysate, and finally the (more or less) purified nucleic acids, particularly RNA and DNA have to be recovered (an overview can be found in the QIAGEN brochure on “Genomic DNA Purification”). The step of purifying the DNA is of utmost importance, as carryover of mixture or buffer contaminants such as salts, detergents, organic solvents, in particular phenol and ethanol, often inhibit performance of DNA in downstream applications.

A very simple and fast technique for the isolation of genomic DNA from cell lysates is to incubate the cell lysates at high temperatures, e.g. at 90° C. for about 20 min or to directly use the lysates after an additional protease digestion. However, these lysates usually contain enzyme-inhibiting contaminants such as a high salt load, and accordingly these methods, which are considered as quick and dirty techniques, are only appropriate for a limited range of applications.

So-called salting-out methods, wherein proteins and other contaminants are precipitated from the crude cell lysate by adding a solution comprising a high concentration of a salt, such as potassium acetate or ammonium acetate, are well-known techniques for separating DNA from other cellular components present in a cell lysate. The precipitates formed are then removed from the solution comprising the DNA e.g. by centrifugation, and the DNA is recovered from the supernatant usually by precipitation with alcohol in a further step. In these methods, removal of proteins, in particular nucleases, and other contaminants often is quite inefficient, and an additional RNase treatment, a dialysis and/or repeated precipitations by alcohol may be necessary to obtain DNA sufficiently pure to be used in downstream applications, which renders the methods tedious and time-consuming.

Another possibility to separate nucleic acids from the other compounds present in a cell lysate is to extract the contaminants from the lysates using organic solvents. In a first step, the cells typically are lysed using a detergent, and the lysates are then extracted using solvents, such as phenol, chloroform, and isoamyl alcohol, to remove the contaminants. The toxicity of the solvents used is one drawback of these methods. Furthermore, special attention has to be paid to the pH and salt concentration to ensure that the majority of contaminants are extracted into the organic phase, while the nucleic acids, particularly DNA, remain(s) within the aqueous phase. The nucleic acids are then recovered from the aqueous phase by alcohol precipitation. Even though organic extraction methods are very time-consuming, the DNA isolated using these methods often contains residual phenol and/or chloroform, which act as inhibitors in downstream applications such as PCR. In addition, toxic waste is generated which has to be disposed in accordance with hazardous waste guidelines.

In recent years sorption procedures based on ion exchange, affinity and/or hydrophobic interactions have been developed in order to minimize DNA degradation during purification. In these sorption procedures, the DNA is more or less specifically “sorbed”, that is either adsorbed, absorbed or chemically bound, to a stationary solid phase, comprising a resin or a matrix, due to specific interactions between the DNA and the solid phase, while contaminants do not interact with the solid phase to the same extent as DNA does, and thus may be separated from the sorbed DNA, e.g. by a washing step. Once the contaminants have been removed, the DNA has to be recovered from the solid phase by an eluting step, which usually includes a step of rinsing the solid phase with a solution (mobile phase) comprising compounds that minimize the interaction between the solid phase and the DNA, thus removing the DNA from the solid phase. The mobile phase comprising the DNA (eluate) is then collected. These solid phase-based methods enable an automation of the process of DNA isolation and purification. In addition, also rather minute amounts of DNA can be reliably processed using these methods.

Anion-exchange methods are based on the interaction between the negatively charged phosphates of the nucleic acids and positively charged surface molecules on the anion-exchange carrier (Forcic et al., J. Chromatogr. A 2005, 1065(1), 115-120). Under low-salt conditions DNA present in solution selectively binds to the stationary phase, and impurities such as RNA, cellular proteins, and metabolites may be washed away from the stationary phase using medium-salt buffers. In the next step, DNA can be eluted from the stationary phase using a buffer containing a high concentration of salt. The purified DNA is then recovered from the eluate by alcohol precipitation.

In silica-based methods, nucleic acids are selectively sorbed to a silica-gel membrane in the presence of high concentrations of chaotropic salts (Hanselle et al. Leg Med (Tokyo) 2003, 5 Supp. 1, 5145-5149). RNA, cellular proteins, and metabolites are washed away from the membrane, and the DNA is then eluted from the silica-gel membrane using a low-salt buffer.

Also solid-phase methods based on the interaction between DNA and magnetic particles as a stationary phase are known in the state of the art (Prod\check{e}lalová et al. J. Chromatogr. A 2004, 1056, 43-48).

Even though sorption methods allow the isolation of high-quality nucleic acids, the number of steps to be carried out in these “bind-wash-elute” routines still is comparatively high and thus time-consuming. For this reason a need exists for a fast and gentle method of isolating nucleic acids, preferably DNA and RNA, more preferred selective isolation of either DNA and RNA or highly purified DNA (free of RNA), from biological samples, such as tissue and blood, wherein the number of steps to obtain the purified nucleic acids is reduced in comparison to the known sorption procedures, such as anion-exchange and silica-based methods, without compromising the stability or purity of the nucleic acids obtained. Such a method should enable the user to reliably lyse biological samples, and to isolate and purify the nucleic acids present in the obtained lysate from contaminants such as proteins, in particular nucleases, lipids, and other cellular constituents. On the other hand the method should be gentle enough to minimize thermal, chemical or enzymatic degradation of the nucleic acids and mechanical shear stress, which otherwise would fragment particularly the large genomic DNA during the course of purification. In addition, the method should offer the user the possibility to select which type of nucleic acids should be isolated and should provide the possibility to accommodate a wide variety of biological samples of different origin.

According to the present invention the term “nucleic acids” comprises any type or DNA or RNA as well as a mixture of DNA and RNA of any type. The term “selective isolation” refers to the possible option either to isolate in one procedure DNA and RNA (combined), or to isolate highly purified DNA, which means that RNA as well is separated from the DNA, resulting in a sample wherein essentially no RNA is contained.

It was an object of the present invention to provide a method for selective isolation and purification of nucleic acids, particularly comprising DNA from a cell-containing biological sample, wherein the number of steps required to isolate the nucleic acids, purified from contaminants such as proteins, in particular nucleases, and other cell components is reduced in comparison to the known methods, while still ensuring high-quality nucleic acids.

It has now been found that high-quality nucleic acids selectively can be obtained from a variety of biological samples by a gentle method for isolating and purifying nucleic acids from a biological cell comprising sample comprising at least DNA, RNA, and proteins, comprising at least the steps of 1. mixing the sample with a lysis buffer, 2. incubating the sample at a temperature either within a range between 45° C. and 59° C. to obtain DNA as well as RNA or within a range between 60° C. and 70° C., to obtain DNA essentially free of RNA and 3. separating the nucleic acids from any (further) contaminants. According to the present invention the isolation or purification method for either isolating DNA and RNA or for isolating DNA essentially free of RNA is incidentally the same, which means that all the steps for isolation or purification correspond to each other except the temperature applied to the sample.

According to the present invention it has been found, that it is possible to differ between the isolation of DNA as well as RNA from the same sample and the isolation of DNA essentially free of RNA by lysing the sample at different temperature ranges. It was surprisingly found that after lysis within a temperature range of up to 59° C. RNA remains in the cell lysate side by side with the DNA, whereas increasing the temperature to a range of 60° C. or higher results in almost full RNA degradation, so that a sample is obtainable comprising essentially no RNA. This finding provides the option for a person isolating nucleic acids by any suitable method to select whether DNA and RNA shall be isolated as a mixture from the same sample or whether only DNA is of interest. Accordingly it is only necessary to keep the temperature conditions within the appropriate range using incidentally the same method. Preferably for isolation of RNA and DNA the temperature conditions are selected within a range of 50° C. to 59° C., more preferred 54° C. to 58° C., particularly preferred of 55° C. to 57° C. and most preferred of 56° C., whereas for isolation of DNA essentially free of RNA the temperature is preferably within the range of 61° C. to 65° C., more preferred 61° C. to 63° C., particularly preferred 62° C. Accordingly the temperature range used at least in the lysis step or after lysing the cells during the isolation method can be used to select whether DNA free of RNA shall be isolated or a mixture of DNA with (optionally at least partially degraded) RNA. Preferably no additional step for degradation of RNA has to be carried out, in particular no RNase has to be added.

The selected temperature range of 45° C. to 59° C. or of 60° C. to 70° C. can be used during an incidentally similar or even identical nucleic acid isolation and/or purification method to differ between the isolation of a DNA/RNA mixture or DNA essentially free of RNA from a cell-containing biological sample, preferably the same (type of) sample in both cases.

The period of time a sample has to be heated in order to ensure complete lysis and optionally RNA disintegration depends on the kind and amount of sample being processed and in some cases from the used lysis buffer. Preferably the mixture is heated for at least 5 min, e.g. 10 to 80 minutes (min), more preferably for 15 to 60 min, even more preferably for 20 to 50 min, and most preferably for 30 to 45 min. It is particularly preferred that the appropriate temperature range is used during lysis of the cells or the sample after lysis (i.e. the lysate) is incubated for at least 5 min at the according temperature. It is notable that if RNA shall remain in the isolated nucleic acids, the temperature should not exceed 59° C. during the isolation procedure, at least not as long as the cell proteins are present in the sample. If RNA shall remain in the isolated nucleic acids it is particularly preferred that the temperature during the whole isolation or purification procedure doesn't exceed 59° C.

Essentially any suitable method for isolating and purifying nucleic acids from a cell comprising biological sample can be adapted to the selective method according to the present invention. Particularly any method comprising a lysing step of the biological cell comprising sample can be varied within the above mentioned temperature range for selection of the obtainable nucleic acids. In a preferred embodiment a method for isolation and purification of nucleic acids is used in which the nucleic acids remain essentially in solution during the whole isolation procedure. This means that no step for deliberate precipitation or sorption or bonding of the nucleic acids is carried out during the procedure. In another preferred embodiment any method is used in which the nucleic acids after lysis of the biological cell comprising sample are contacted with any “non-selective” solid matrix, resulting in sorption, bonding or retaining of the nucleic acids at or within the matrix. “Non-selective” means here, that the matrix is not selective for one particular type of nucleic acids, but sorbes, binds or retains any type of nucleic acid. In still another embodiment the isolation method as well can comprise a step of “non-selective” precipitation of nucleic acids, resulting in a precipitate comprising either a mixture of (optionally partially degraded) RNA and DNA, or DNA essentially free of RNA.

The term “essentially free” means that the RNA originally comprised in the lysed sample is separated or decomposed to an amount of least 50%, preferably at least 60%, more preferred at least 75% and most preferred at least 90%, particularly preferred 95% compared to the original amount of the biological sample. To obtain highly purified DNA an additional step of RNA degradation, e.g. by addition of RNAse is optionally included in the method, independent from the temperature range of 60 to 65° C.

For lysis of the cells any of the known suitable lysis buffers can be used without any restriction as far as the lysis buffer itself doesn't degrade RNA or DNA. In a particularly preferred embodiment for the lysis a lysis buffer is used comprising an anionic surfactant, but preferably being essentially free of a chelating or complexing agent. Such a lysis buffer is described in detail in the co-pending patent applications with the titles “method for isolating and purifying nucleic acids” and “method for precipitating anionic surfactant ions in the presence of nucleic acids” of the same applicant having the same filing date of the present application.

If said preferred embodiment is used, it is further preferred to include in the method the following steps: (i) precipitating the surfactant ions from the lysate by adding to the lysate a solution comprising monovalent ions of alkali metals and/or divalent ions of alkaline earth metals selected from the group comprising, preferably consisting of Rb⁺, Cs⁺, Ca²⁺, Sr²⁺, Ba²⁺, or a mixture thereof, (ii) separating the nucleic acids from the precipitate and further contaminants present in the lysate by size-exclusion chromatography to obtain a purified nucleic acid-containing eluate. The nucleic acids, particularly the DNA preferably remain essentially in solution during all of the method steps.

Using said preferred embodiment of the method of the present invention highly purified nucleic acids can be obtained, e.g. from tissue samples in about only 45 min (30 min lysis, 10 min of precipitation, 3 min pre-spinning of the column and optionally pelleting the precipitate (may be carried out at the same time) and 3 min for the chromatographic separation itself), while approximately 2.5 h typically are necessary for the lysis and purification of the same amount of tissue using e.g. the QIAamp kit (QIAGEN, Hilden, Germany). The method of the present invention provides in any case purified nucleic acids comprising DNA, purified from cellular contaminants. Particularly, dependent from the conditions and the steps used, either a mixture of DNA and RNA is obtainable or highly purified DNA can be prepared, which is as well essentially separated from RNA. If in the following the term “DNA” is used, the DNA-containing purified nucleic acid sample is meant, either comprising RNA, or separated from RNA as well. Preferably the conditions of the method are resulting in a highly purified DNA, comprising essentially no RNA.

Due to the gentle temperature conditions the quality of the nucleic acids, particularly of the DNA, isolated by the selective method of the present invention is equal, or in many cases even superior, to the quality of nucleic acids obtained by state of the art methods for bench-scale purification, such as for example the very successful QIAamp technology (QIAGEN, Hilden, Germany), particularly with respect to the yield as judged by UVN is spectroscopy, gel electrophoresis, conductivity measurements, HPLC analysis, PCR and further assays. The purity of the obtained nucleic acids, particularly the purity of the DNA in any case is comparable to the purity of the nucleic acids as isolated by the according “base method”. The “selection step” by adapting the temperature conditions can be included in nearly all known nucleic acid methods. Furthermore, the selection step can be included in any method which can be fully automated.

By adapting the temperature conditions to the selected temperature range, in principle all kind of desoxyribonucleic acid (DNA) and all kind of ribonucleic acids (RNA) can be isolated from a wide variety of biological samples, including synthetic, genetically engineered or naturally occurring single-stranded or double-stranded DNA, oligo- and polynucleotides of desoxyribonucleotides, fragments of DNA obtained by partly digesting DNA using restriction endonucleases, mitochondrial DNA, plasmid DNA, and metagenomic DNA, single or double stranded RNA like e.g. mRNA, tRNA, rRNA, snRNA, hnRNA, snoRNA, siRNA or ribozymes, representing the entirety of DNA or RNA obtained from all microorganisms found in a biotope or a biocenosis. Preferably the method of the present invention is used for isolating and purifying any type of DNA, particularly including genomic DNA, which in terms of the present invention is the high molecular weight DNA obtained from one single organism, comprising the entirety of genetic information of this organism, in contrast to plasmid DNA, DNA partly digested by the action of restriction endonucleases, and metagenomic DNA. For example in the above mentioned preferred embodiment, purified high molecular weight DNA is obtained, while smaller fragments of DNA are retained within the chromatographic material. Due to its high molecular weight and large size, intact high quality genomic DNA is difficult to isolate and purify, as a comparably high risk of degradation of genomic DNA exists, either by mechanical stress during the isolating procedure, in particular sheer stress, or by chemical and enzymatic degradation. Degraded DNA, on the other hand, may lead to both quantitative and qualitative errors in downstream analyses. The method of the present invention provides a fast, robust, safe, easy-to-handle and yet mild method for isolating and purifying nucleic acids, particularly containing DNA, in particular genomic DNA, from a variety of different biological samples.

It has been found that a fast, yet mild lysis of biological samples can be achieved by using a new lysis buffer comprising an anionic surfactant providing a surfactant ion, preferably an anionic surfactant providing a source of sulfate ions, most preferably dodecyl sulfate ions (DS⁻), but preferably being essentially free of a chelating or complexing agent. Said lysis buffer is an aqueous solution, comprising active components such as detergents, to disrupt and/or break the cellular membrane of a cell, causing the intracellular components, such as DNA, RNA, proteins, lipids, metabolites etc., to be released into solution. The solution comprising the former intracellular components is called lysate.

In a preferred embodiment the lysis buffer comprises a buffering substance, H₂SO₄ and an anionic surfactant, wherein the buffer has a pH of 7.5 to 10, preferably of 8 to 9 and most preferably of 8.5 and is preferably essentially free of any chelating or complexing agent and Mg²⁺-ions.

Such a buffer allows a fast lysis of sample material under low-salt lysis conditions. The term “low salt conditions” refers to hypotonic conditions, which means that the total ion concentration in the buffer solution is lower than the total ion concentration within the cells to be lysed. In the case of NaCl, for example, an aqueous solution comprising less than 0.9 wt % NaCl (about 155 mmol NaCl, corresponding to about 310 μmol/L of dissolved ions) is hypotonic. Even samples containing a rather high amount of solid material, for example tissue samples, are usually completely lysed within less than 40 min at e.g. 56° C. In principle, lysis can be carried out at temperatures ranging from 45° C. to 70° C., preferably from 50° C. to 68° C., and most preferred at 62° C., dependent from the desired nucleic acids. Since the preferred lysis buffer comprises a rather low amount of salt and preferably is essentially free of a chelating or complexing agent for the divalent ions necessary as co-factors for the polymerase in PCR reactions, and the pH of the lysis buffer falls within the optimum pH range for PCR reactions, the lysate obtained using such a lysis buffer can be directly used in downstream applications such as for example qRT-PCR. Details of a suitable lysis buffer can be found in the co-pending application with the title “method for isolating and purifying nucleic acids” from the same applicant having the same filing date as the present application.

According to the present invention, RNA present in the obtained lysate may be optionally or additionally disintegrated after lysis of the sample. In terms of the present invention the step of disintegrating RNA comprises any method of reducing the amount of dissolved RNA in the lysate and/or inactivating the RNA and/or facilitating its separation from the DNA, including any method of thermally, chemically and/or enzymatically hydrolyzing, digesting, transforming and/or so decomposing RNA, either partially or completely, and/or removing the RNA or its fragments from the solution, e.g. by precipitation, sorption procedures or the like. Basis of the present invention is that one simple method for disintegrating the RNA in the sample is by heating the sample to a temperature of at least 60° C. without any further addition of a disintegrating agent. If the RNA shall remain in the sample heating of the sample only up to 59° C., preferably up to 58° C., more preferably up to 56° C. is recommended. If highly purified DNA (essentially free of RNA) is desired, the step of incubating the mixture of the biological sample and the lysis buffer, and the optional step of disintegrating the RNA present in the lysate are carried out in a single step, preferably by heating the mixture to a temperature equal to or above 60° C., preferably to 60° C. to 70° C., more preferably to 61° C. to 65° C., and most preferably to 62° C. The period of time a sampie has to be heated in order to ensure complete lysis and RNA disintegration depends on the kind and amount of sample being processed. Preferably the mixture is heated for at least 5 min, e.g. 10 to 80 minutes (min), more preferably for 15 to 60 min, even more preferably for 20 to 50 min, and most preferably for 30 to 45 min.

The volume of lysis buffer used to lyse a certain amount of cell-containing sample material depends upon the kind and size of the sample material being processed and the amount of the matrix in the gel filtration device. In the case of tissue samples it may for example be useful to cut large tissue sample into pieces of approximately 4 mm³ or less, before mixing the sample with the lysis buffer. Preferably a lysis buffer volume of 20 to 150 μL, more preferably of 30 to 120 μL, even more preferably of 50 to 100 μL and most preferably of 80 μL is used for the lysis of 10 mg of sample tissue. Preferably the amount of the matrix to purify said lysate should be 500 μl to 1000 μl and more preferably 600 μl to 900 μl and most preferably 800 μl. The matrix is preferably provided as a dispersion of the gel-forming polymer in water, a salt solution, e.g. 0.9% NaCl, or a suitable buffer, like e.g. TE, TAE, PBS or similar or in diluted buffers, whereas said dispersion comprises preferably 60-90%, more preferably 70-80% and in particular 75% of the gel-forming polymer. The amount of DNA obtainable from 10 mg of a sample using the method of the present invention depends upon the sample, for example its kind and age. Usually around 5 to 70 μg genomic DNA are obtained from 10 mg of different tissue samples using the method of the present invention.

Amounts of about 10 mg are the amount of sample commonly analyzed in molecular diagnostics. It should, however, be understood, that using the method of the present invention, it is also possible to process larger or smaller amounts of sample material, e.g. in the g-range or μg- to ng-range, respectively. In this case, the amounts of reagents, buffers, solid matrix as well as the dimension of the chromatographic device, if used, have to be adjusted by up- or downscaling, which is well known to a person skilled in the art.

By using the “selection step” in any nucleic acid isolation or purification method cells from a wide variety of biological samples can be lysed and further processed in order to purify the nucleic acids contained therein, preferably comprising genomic DNA, including, but not limited to, animal and human tissue, for example liver, spleen, lung, heart, brain, kidney etc., animal and human blood, cell cultures of animal and human cells, animal and human bone marrow, liquor, sputum or sperm, yeast, bacteria, insects, plants, and rodent tails. Preferably, the samples are cell-containing biological samples of animal or human origin. In another preferred embodiment, the samples comprise or consist of Gram-negative bacteria. The sample may be lysed immediately after having been taken from its natural environment (fresh sample), or may be stabilized prior to lysis by freezing or by the action of chemical stabilizing agents, such as for example formalin-fixing and paraffin-embedding (FFPE tissue) or blood stabilizing agents comprising citrate or Heparin. Even more preferably the sample is selected from the group comprising fresh or frozen tissue and blood, most preferably from mammalian tissue and blood.

The present invention accordingly provides a nucleic acid isolation and/or purification method including mixing a cell-comprising biological sample comprising at least DNA, RNA and proteins with a lysis buffer and adapting the temperature range used during lysis either to 45° C. to 59° C. or to 60° C. to 70° C. to obtain either a DNA/RNA mixture or to obtain DNA essentially free of RNA, whereas all the other steps of the isolation/purification method are incidentally the same.

As mentioned above further purification of the nucleic acids comprised in the obtained lysate can be carried out by any known “non-selective” nucleic acid purification method, either resulting in a RNA/DNA mixture or in DNA essentially free of RNA, dependent only by the temperature conditions selected in the “selective step” as described above.

If the above described preferred lysis buffer is used for lysis of the biological cell containing sample and further the surfactant ions are precipitated as described in the co-pending application of the same applicant having the title “method for isolating and purifying nucleic acids” of the same filing date, nucleic acid purification, particularly separation of the nucleic acids from the precipitate and further contaminants present in the lysate, preferably can be carried out by a chromatographic device for purifying nucleic acids, from contaminants by gel filtration chromatography as described in co-pending application with the title “chromatographic device and method for isolating and purifying nucleic acids” of the same applicant having the same filing date as the present application. Such a chromatographic device preferably comprises at least one chromatographic unit, comprising: 1. A hollow body having an inlet and an outlet, the hollow body comprising a solid matrix providing size excluding properties, preferably forming a gel bed; 2. a porous frit, filter, fleece or membrane preferably allowing nucleic acids of any size to pass, placed between the outlet and the solid matrix to retain the solid matrix within the chromatographic unit, 3. optionally a non-porous ring placed between the porous frit, filter, fleece or membrane and the matrix, sealing the outer area of the frit, filter, fleece or membrane, to prevent the mobile phase from entering the frit without passing the matrix, 4. optionally at least one removable closing device to seal the inlet and/or outlet of the chromatographic unit, and 5. optionally at least one collection tube to collect the mobile phase (eluate) after having passed the matrix, wherein the solid matrix in a preferred embodiment is a gel forming polymer having a size exclusion limit of 150 to 500 base pairs (bp), preferably 200 to 400 bp, and most preferably 250 to 300 bp. Preferably the gel forming polymer has a corresponding size exclusion limit of 10 to 10.000 KDa, more preferred of 20 to 8.000 kDa.

Such a chromatographic device preferably is used for size exclusion chromatography (SEC). Preferably a water-based mobile phase, such as water, an aqueous organic solvent or an aqueous buffer/solution, is used as mobile phase. In this case SEC is also referred to as gel filtration chromatography. Size exclusion chromatography is a chrometographic method, wherein molecules are separated based on their size, or more precisely based on their hydrodynamic volume. Commonly, a solid matrix able to form a gel bed, when suspended in an aqueous medium, such as a dextran, agarose, polyacrylamide, or a mixture thereof, is suspended in a buffer and packed in the hollow body of a column made of glass, plastic, Teflon or any other material that neither reacts with the mobile phase nor the analyte. The sample to be purified is then applied to (the center of) the gel bed's upper surface, and allowed to pass through the gel, either by gravity or forced by centrifugation, vacuum or pressure. According to the present invention preferably centrifugal forces are applied to move the mobile phase down the column, wherein the columns are spun in a centrifuge (so-called spin column technique). Due to the cross-linking in the gel, pores of a certain size exist inside the gel. Small molecules are able to penetrate the pores, and therefore move through the gel bed more slowly, being retained as they pass down the column, while large molecules cannot penetrate the pores and move down the column more quickly. After having passed the column, the mobile phase (now referred to as eluate), containing the purified analyte, is then collected at the outlet of the column. To retain the solid matrix within the hollow body of the column, a porous frit, filter, fleece or membrane is preferably placed between the outlet of the column and the solid matrix, wherein nucleic acids of all sizes may pass said frit, filter, fleece or membrane.

In SEC, the size exclusion limit defines the molecular weight, where molecules are too large to be trapped in the stationary phase. The size exclusion limit of a solid matrix can be adjusted by the degree of cross-linking in the gel. A wide variety of solid matrices able to form a gel bed with different degrees of cross-linking are commercially available.

The present invention is also represented by a kit for isolating or purifying nucleic acids from cell-comprising biological samples, comprising 1. a lysis buffer, 2. optionally a chromatographic device as described herein and 3. instructions to carry out incidentally the same isolation or purification method either at a lysis temperature in the range of 45° C. to 59° C. or in the range of 60° C. to 70° C. dependent from the desired nucleic acids, which either result in DNA/RNA mixture or in DNA essentially free from RNA, respective from the applied temperature range.

### DESCRIPTION OF THE DRAWINGS

FIG. 1 shows a SYBR-Green II stained agarose gel obtained by gel electrophoresis of pork liver tissue samples, lysed with a buffer comprising TRIS, SDS, EDTA and MgCl₂ according to reference example 2. A DNA size standard is shown in lane 1. The samples analyzed in lanes 2 and 3 have been purified using the QIAsymphony platform (QIAGEN, Hilden), while in samples shown in lanes 4 and 5 as well as 6 and 7, dodecyl sulfate ions were removed by the addition of K₂CO₃ and KHCO₃, respectively. A large amount of ribosomal RNA (rRNA) is present in all the samples treated with potassium ions.

FIG. 2 is a picture of an agarose gel obtained from lysates of 10 mg pork liver tissue after having been incubated at different temperatures for 40 min (see Example 3). While in the sample incubated at 56° C. RNA still is clearly visible, disintegration of RNA is possible by increasing the incubation temperature. The picture on the right hand side furthermore shows, that the integrity of the gDNA is not compromised, particularly when no EDTA is present in the lysis buffer (see Example 3).

FIG. 3 shows an agarose gel analysis of lysates of 10 mg liver tissue, after having been incubated for 30 min at 62° C. in different buffers or aqueous solutions (see Example 3).

### EXAMPLES

### Materials and General Experimental Procedures

Gel filtration media were obtained from GE-Healthcare (Freiburg, Germany), ion-exchange media were obtained from Merck KgaA (Darmstadt, Germany).

Unless otherwise noted, the tissue samples analyzed were rat liver tissue samples.

Determination of the amount and purity of gDNA: To estimate the amount of gDNA (gDNA yield) in a purified so sample (eluate), the absorbance of the sample was measured at a wavelength of 260 nm by UVN is spectroscopy. A background absorption value, measured at 320 nm was subtracted from the 0160 value (optical density at 260 nm), and the value was multiplied by 50, the specific absorbance factor of DNA, and by the dilution factor to obtain the gDNA concentration in μg/μL. In addition, UVN is spectroscopy was also used to judge the purity of the DNA obtained. Residual solid particles do not exhibit a distinct absorbance peak, but lead to an elevated baseline in the whole spectrum. Free haemoglobin has an absorbance maximum at a wavelength of 410 nm, while salts and preservatives like sodium azide absorb at a wavelength below 230 nm. A Spectramax II (Molecular Devices, Sunnyvale, Calif., USA) 96-well plate photometer was used to record the UVN is spectra.

A more precise determination of the amount of gDNA obtained was carried out using HPLC analysis. The area under curve (AUC) for the gDNA-containing peaks in the spectra was calculated by the software and compared to a HPLC standard curve, to determine the amount of gDNA in a sample. HPLC analysis was also used to determine the purity of the samples using a Vision BioCad workstation (Perseptive Biosystems, Framingham, Mass., USA). A 0.83 mL Peek column filled with the ion exchange resin TMAE-Fractogel(S) (E. Merck, Darmstadt, Germany) was used. The samples were analyzed at a flow rate of 1.5 mL/min in an increasing CaCl₂ gradient, starting from 0 mmol/L to 300 mmol/L over a period of 35 column volumes, buffered at pH 7.2. The absorbance was continuously monitored at 260 nm and 410 nm.

Agarose gel electrophoresis was carried out using a 50 mL 0.8% agarose gel, containing 2.5 μL SYBR-Green II. Samples were run using a voltage of 100 Volt for a time period of 40 min. The gels were analyzed using commercially available equipment from BioRad or LTF-Labortechnik (Wasserburg, Germany).

SDS quantification: The residual SDS concentration was determined by UVN is spectroscopy according to a modified procedure of Rusconi et al. adapted to be used within a 96 well photometer (Rusconi et al. Anal. Biochem., 2001, 295(1), 31-37). The assay is based on a specific reaction of the carbocyanine dye “Stains All” (4,5,4′,5-Dibenzo-3,3′-diethyl-9-methylthiocarbocyanine bromide) with SDS, which leads to the formation of a yellow colour (absorbance maximum at 438 nm). As SDS was used as the source of dodecyl sulfate ions in the present examples, it should be understood that the amount (molarity) of SDS in a solution equals the amount (molarity) of dodecyl sulfate ions present in solution.

1 mL of a stock solution of the dye (1.0 mg “Stains All” in 1.0 mL 50% isopropanol) was diluted with 1.0 mL formamide and 18 mL water to obtain a ready-to-use solution of the dye. To determine the amount of SDS in a sample, 5 μL of the sample solution were placed into a microtiter plate, mixed with 100 μL of the ready-to-use solution, and incubated at room temperature for 5 min in the dark before reading the plate at 438 nm. The amount of SDS in the sample was retrieved by comparison with a calibration curve, established by recording the absorbance of solutions containing a SDS concentration of 250, 167, 111, 74, 49, 32 and 21 μmol/L, respectively, at 438 nm.

Conductivity measurement: To determine the ion strength in the samples, conductivity measurements were carried out using a Consort C831 Conductometer (LTF-Labortechnik, Wasserburg, Germany), calibrated to 20° C. A minimum volume of 2 mL is necessary for the measurement, therefore aliquots of 20 μL of each sample were diluted with 1980 μL water prior to the measurement.

PCR amplifications: Real time-PCR (qRT-PCR) assays were performed on a Rotor-Gene 2000 or 3000 cycler (Corbett, Sydney, Australia) on a 50 μL scale, or in a TagMan 7700 analyzer (Applied Biosystems, Foster City, Calif., USA).

For the jun RT-PCR assay, a commercially available kit (Part. No: 4327113F), based on a primer/probe system (FAM) from Applied Biosystems (Darmstadt, Germany), including a 20× Jun PCR primer/probe mix was used in combination with a 2× TagMan PCR universal master mix from Applied Biosystems.

A genomic DNA standard was purified from rat tail using the QIAsymphony DNA kit on QIA-symphony platform (QIAGEN, Hilden, Germany), and was further purified by subsequent anion exchange chromatography (AEX) using a QIAGEN tip 2500 according to the manufacturers' protocol (QIAGEN, Hilden, Germany). The gDNA was stored in aliquots at −20° C. and thawed immediately prior to use.

### Example 1

### Lysis of Pork Liver Tissue with Different Buffers, Different pH

Lysis buffers of the following compositions were prepared: reference buffer A: 100 mmol/L TRIS, 5 mmol/L EDTA, 100 mmol/L MgSO₄ and 100 mmol/L SDS, adjusted to pH 6.0 by the addition of H₂SO₄; reference buffer B: 100 mmol/L TRIS, 5 mmol/L EDTA, 100 mmol/L MgSO₄ and 100 mmol/L SDS, adjusted to pH 8.0 by the addition of H₂SO₄; lysis buffer according to the present invention: TRIS 25 mmol/L, SDS 25 mmol/L, adjusted to pH 8.5 by the addition of H₂SO₄ (25% v/v). All buffer compositions were prepared as aqueous solutions. Reference buffer compositions A and B are based on standard buffer compositions, commonly used for lysing cell-containing material, additionally containing Mg²⁺-ions, which were reported to degrade RNA at alkaline pH and temperatures above 37° C. (N. G. AbouHaidar and I. G. Ivanov Z. Naturforsch. 1999, 54 c, 542-548). Samples of pork liver tissue (25 mg each) were incubated with 500 μL of reference buffer A, reference buffer B and the buffer according to the present invention, respectively, at 56° C. To aid depletion of proteins 10 μL of QIAGEN Proteinase K (2.5 AU/ml) (QIAGEN, Hilden, Germany) was added to each sample solution. While lysis of the pork liver tissue was usually completed within 40 min having used reference buffer B and the buffer of the present invention, residual tissue fragments were still present even after two hours of incubation when having used reference buffer A. Thus, the results described by AbouHaidar et al. could not be verified.

### Reference Example 2

### Precipitation of Dodecyl Sulfate Ions from the Lysate Obtained Using Reference Buffer B

Although it was known from the state of the art that potassium salts of dodecyl sulfate have a very low solubility at acidic or almost neutral pH, it was initially attempted to precipitate the dodecyl sulfate ions in the lysate obtained using reference buffer B by the addition of potassium ions. With the intention to directly use the lysates in a PCR reaction, after precipitation of dodecyl sulfate ions, and subsequent removal of the precipitate formed, solutions of alkaline potassium salts were added to the lysate in order to precipitate dodecyl sulfate ions, as PCR reactions usually require a pH of about 8 to 9.

For this reason potassium carbonate (K₂CO₃) and potassium bicarbonate (KHCO₃) were tested as an alkaline source of potassium ions. Samples of 20 mg of pork liver tissue were suspended in 500 μL of reference buffer B (pH 8.0) and incubated at 56° C. for 30 min. To precipitate the dodecyl sulfate ions from the lysate, 140 μL of a 0.25 M aqueous solution of K₂CO₃ per sample and 350 μL of a 0.25 M aqueous solution of KHCO₃ per sample were added to two samples, respectively. All samples were incubated in an ice bath for 5 min. While the addition of KHCO₃ did not alter the pH of the lysate significantly, a slight increase of pH was observed after addition of K₂CO₃. For this reason samples treated with K₂CO₃ were neutralized by adding 3 μL 2% HCl (aq). All samples were centrifuged to remove the precipitate, and aliquots of the supernatants (4 μL) were analyzed on an agarose gel. As a positive control DNA from two further samples of pork liver tissue lysed using reference buffer B was purified on a QIAsymphony platform (QIAGEN, Hilden, Germany) according to the QIAsymphony DNA handbook 05/2008, using a bind-wash-elute concept based on magnetic particles including a RNAse treatment.

The electrophoresis gel is shown in FIG. 1. The Gibco 1 kb Plus DNA Ladder (Invitrogen GmbH, Karlsruhe, Germany)) was used as a length standard to identify the size of fragments present in the samples (lane 1). In lanes 2 and 3, lysates purified using the QIAsymphony platform were analyzed. In these samples mainly gDNA of a decent quality is detected, while a large amount of ribosomal RNA (rRNA) is present in the samples treated with K₂CO₃ (lanes 4 and 5) and KHCO₃ (lanes 6 and 7). The presence of rRNA in the samples was unexpected, since it should have been hydrolyzed by the combination of a TRIS buffer and magnesium ions within a lysis time of 30 min at pH 8.0 according to AbouHaidar.

The lysis experiment was therefore repeated using different concentrations of reference buffer B, ranging from a 0.25-fold concentration to 2-fold concentration with an increased incubation time of 40 min at 56° C. Regardless of the buffer concentration, intact RNA was detected in the SYBR-green II stained agarose gel of all samples (data not shown). Accordingly, a combination of magnesium ions with a TRIS buffer additionally containing SDS at a slightly alkaline pH cannot be used for the disintegration of RNA from lysed tissue samples in the presence of intact genomic DNA.

### Example 3

### Disintegration of RNA at Various Temperatures in the Absence of Magnesium Ions

To study the effect of temperature on the lysis of tissue samples and on the disintegration of RNA present in the lysates obtained, samples of 10 mg pork liver were suspended in a buffer containing 25 mmol/L TRIS and 25 mmol/L SDS, but no EDTA and no MgCl₂, adjusted to pH 8.0 by the addition of H₂SO₄. The samples were incubated at different temperatures for 40 min, and subsequently analyzed by gel electrophoresis.

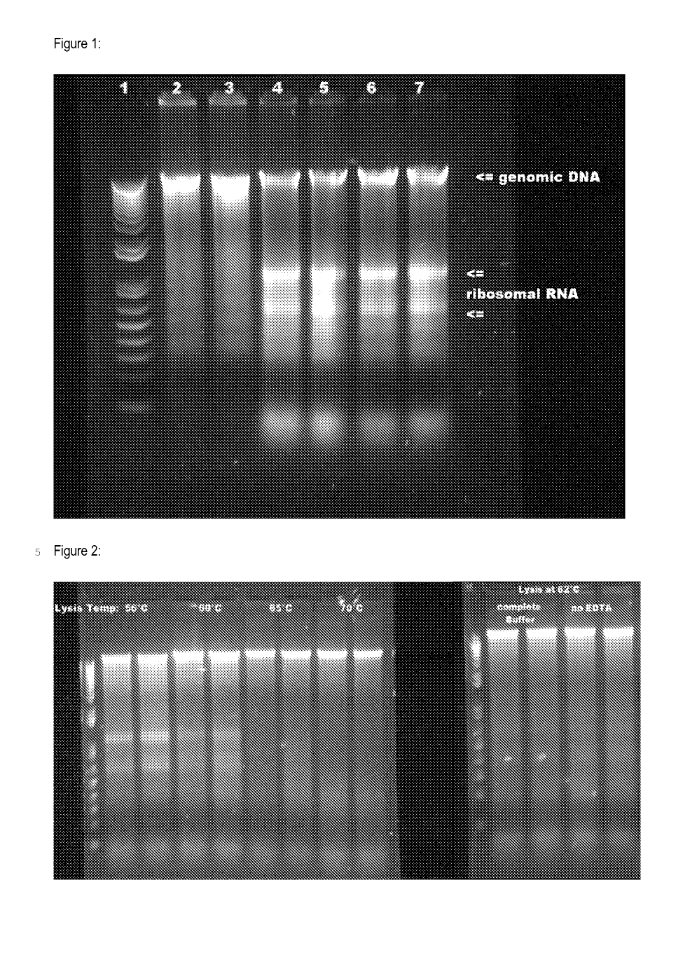

The results are presented in FIG. 2: while a large amount of RNA still was present after having incubated the samples at 56° C., the amount of RNA was significantly reduced after incubation of the samples at 60° C. When the samples were incubated at temperatures above 60° C., no RNA could be detected by gel electrophoresis. The optimum temperature was determined to be 62° C. Furthermore, it was possible to omit EDTA from the lysis buffer without compromising the integrity of gDNA as shown in the agarose gel on the right hand side, which enabled a subsequent PCR reaction without the need for removing the chelating agent.

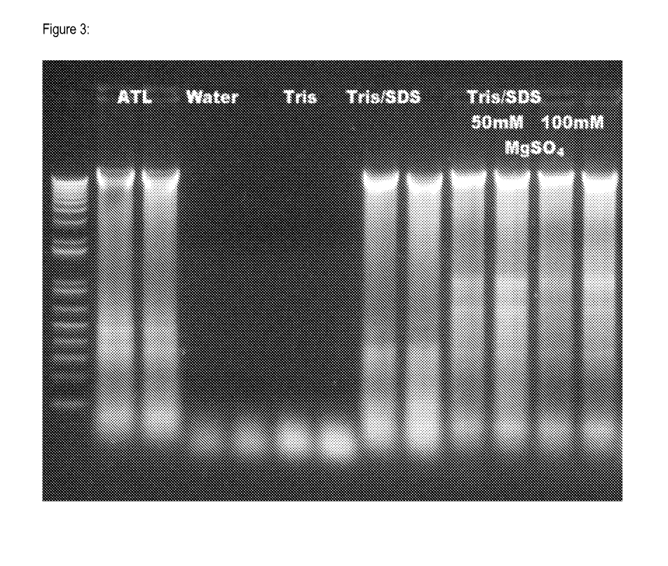

In a further experiment, samples of 10 mg liver tissue were subjected to lysis by incubating the samples in different solutions/buffers at 62° C. for 30 min. Each experiment was carried out in duplicate. The results are presented in FIG. 3: In water as well as in an aqueous solution of 25 mmol/L TRIS, not comprising SDS, no detectable gDNA was so observed. In commercially available QIAGEN ATL buffer (QIAGEN, Hilden, Germany) as well as in a buffer according to the present invention, comprising TRIS and SDS, complete disintegration of RNA was observed, while the gDNA present in the sample was protected from hydrolysis. Addition of MgSO₄ (50 mM or 100 mM, respectively) to TRIS/SDS buffer resulted in that RNA was partially protected from hydrolysis even at a temperature of 62° C. Thus, the optimum buffer composition for isolating DNA essentially free of RNA should comprise TRIS and SDS, both at a concentration of 25 mmol/L, the pH of the buffer solution being adjusted to pH 8.5 by the addition of sulfuric acid, but should be free of Mg²⁺-ions and EDTA. A protease such as QIAGEN Protease or QIAGEN Proteinase K (10 μL; 2.5 AU/ml) may be added to the buffer, preferably after resuspension of the tissue material to aid disintegration of proteins.

1.-6. (canceled)

7. A method for isolating and purifying nucleic acids from a cell comprising biological sample comprising at least DNA, RNA, and proteins, comprising:
(a) mixing the sample with a lysis buffer;
(b) incubating the sample at a temperature
(i) within a range between 45° C. and 59° C. to obtain DNA as well as RNA, or
(ii) within a range between 60° C. and 70° C. to obtain DNA essentially free of RNA; and
(c) separating the nucleic acids essentially from any contaminants.

8. The method according to claim 7, wherein incubating in step (b) is carried out for at least 5 min.

9. The method according to claim 7, wherein incubating in step (b) is carried out for 10 to 80 min.

10. The method according to claim 7, wherein incubating in step (b) is carried out for 15 to 60 min.

11. The method according to claim 7, wherein the temperature range is from 50° C. to 58° C. for isolating a combination of DNA and RNA.

12. The method according to claim 7, wherein the temperature range is from 55° C. to 57° C. for isolating a combination of DNA and RNA.

13. The method according to claim 7, wherein the temperature range is from 61° C. to 65° C. for isolating DNA that is essentially free of RNA.

14. The method according to claim 7, wherein the temperature range is from 61° C. to 63° C. for isolating DNA that is essentially free of RNA.

15. The method according to claim 7, wherein the lysis buffer comprises an anionic surfactant.

16. The method according to claim 7, wherein the lysis buffer is essentially free of a chelating or complexing agent.

17. The method according to claim 15, wherein the lysis buffer has a pH in the range of pH 8 to 9.

18. The method according to claim 16, wherein the lysis buffer has a pH in the range of pH 8 to 9.
